(19) United States
(12) Patent Application Publication
(10) Pub. No.: US 2010/0042014 A1
(43) Pub. Date: Feb. 18, 2010

(54) Safety Biopsy Instrument

(76) Inventors: Ilija Djordjevic, East Granby, CT (US); Sushil K. Kanwar, West Hartford, CT (US)

Correspondence Address: ALIX YALE & RISTAS LLP, 750 MAIN STREET, SUITE 1400, HARTFORD, CT, 06103, US

(21) Appl. No.: 12/583,309

(22) Filed: Aug. 18, 2009

Related U.S. Application Data
(60) Provisional application No. 61/189,522, filed on Aug. 18, 2008.

(51) Int. Cl. A61B 10/02

(52) U.S. Cl. 600/567

(57) ABSTRACT

A safety biopsy instrument having a body, a tubular blade with a sharp edge and a protection member engaged with the body such that the protection member and/or body are axially displaceable relative to each other from a position with the protection member concealing the blade edge to a position with the blade edge exposed, and then to a position with the blade edge concealed and the protection member and blade edge axially locked relative to each other. Also, a safety biopsy instrument with a handle, body and removable blade cartridge with a distal opening, a tubular blade having a sharp edge, and a resistance member disposed between a front shoulder and rear wall to bias the blade edge away from the distal opening. Forward movement of the body relative to the handle causes the resistance member to compress between the rear wall and front shoulder and the blade edge to travel distally.

### CROSS REFERENCE TO RELATED APPLICATION

This application claims the benefit of U.S. Provisional Application No. 61/189,522, filed Aug. 18, 2009 for “SAFETY BIOPSY PUNCH”, the disclosure of which is incorporated by reference in its entirety.

### BACKGROUND OF THE DISCLOSURE

The present disclosure relates to biopsy instruments, and more particularly, to disposable or semi-disposable biopsy instruments with safety elements.

Disposable biopsy instruments currently on the market typically consist of three main components—a handle or base, an integral tubular blade that is permanently attached to the handle and a removable protective cover. These types of instruments can pose a risk of injury from the sharp cutting edge of the tubular blade in the event of removal or loss of the protective cover.

### SUMMARY

There is a need for a safety biopsy instrument that protects against accidental exposure of the blade edge prior to and immediately after use of the biopsy instrument.

According to one embodiment of the disclosed device, the instrument has an axially extending body with distal and proximal ends. A tubular blade is coaxially engaged with the body. The blade has a sharp edge that extends forward of the body's distal end. A protection member is engaged with the body. At least one of the protection member and blade edge are axially displaceable relative to each other from a position with the blade edge concealed by the protection member to a position with the blade edge exposed, and then to a locked concealed position with the blade edge concealed and the protection member and blade edge axially locked relative to each other.

In another embodiment, a medical instrument has an axially extending body with distal and proximal ends. A tubular blade defines a bore. The blade has a sharp circular edge and is coaxially engaged with the body with the blade edge extending forward of the body's distal end. A protective plug has proximal and distal ends and is disposed within the bore. The plug is configured for axial displacement relative to the blade edge. The plug is manually axially displaceable from an initial position with the plug distal end extending forward of the blade edge to a retracted position with the plug distal end positioned within the bore and the blade edge exposed, to an axially locked position with the plug distal end extending forward of the blade edge.

In yet another embodiment, a medical instrument comprises an axially extending body with distal and proximal ends that is disposed coaxially within a tubular handle. The handle has a helical track with closed proximal and distal ends. A pin extends radially from the body and is positioned within the handle track. A removable blade cartridge is configured for engagement with the body within the tubular handle. The blade cartridge comprises a generally cylindrical hollow housing that defines a cavity. The housing has a distal opening. A front shoulder extends radially into the cavity. A tubular blade is attached to a base. The base defines a rear wall. The blade has a sharp edge that extends forward of the rear wall. A return member is disposed within the housing and engaged between the rear wall and front shoulder. The return member biases the blade edge from the distal opening. The distal end of the body is configured to engage the cartridge base. The blade edge is positioned rearward of the distal opening in the housing when the pin is positioned at the proximal end of the helical track. Rotation of the handle in a first direction relative to the base compresses the return member between the rear wall and front shoulder. Compression of the return member causes the blade edge to travel distally to an exposed position forward of the distal housing opening until the pin meets the distal track end. Reverse rotation of the handle relative to the base causes the blade edge to travel proximally to a retracted position rearward of the proximal opening until the pin meets the proximal end of the track.

### BRIEF DESCRIPTION OF THE DRAWING

Aspects of the preferred embodiment will be described in reference to the Drawing, where like numerals reflect like elements:

FIG. 1 shows a typical biopsy instrument with removable cover as known in the art;

FIG. 2 shows an embodiment of the disclosed safety biopsy instrument;

FIG. 3 is an enlarged view of a protection sleeve for use with the disclosed safety biopsy instrument;

FIG. 4A is a partial section view showing the protective sleeve of FIG. 3 engaged with the safety biopsy instrument of FIG. 2;

FIG. 5A shows the disclosed safety biopsy instrument and protective sleeve as removed from a sterile pouch prior to use;

FIG. 5B shows the safety biopsy instrument of FIG. 5A with the blade exposed after rotation and proximal displacement of the protective sleeve;

FIG. 5C shows the safety biopsy instrument of FIGS. 5A and 5B with the protective sleeve locked in place concealing the blade after rotation and distal displacement;

FIG. 6A depicts another embodiment of the disclosed safety biopsy instrument having a safety plug;

FIG. 6B is a section view of the safety biopsy instrument of FIG. 6A;

FIG. 6C is a section view of the safety biopsy instrument of FIGS. 6A and 6B with the blade exposed after retraction of the safety plug;

FIG. 6D is a section view of the safety biopsy instrument of FIGS. 6A, 6B and 6C with the safety plug extended distally past the blade edge;

FIG. 7 is an enlarged view of the blade and safety plug showing detail of the plug's locking lip prior to engagement with the blade edge;

FIG. 8 is an enlarged view of the blade and safety plug showing detail of the plug's locking lip engaged with the blade edge;

FIG. 9 is a section view of another embodiment of the safety biopsy instrument with a blade ejector;

FIG. 10 depicts a cutting depth setting device for optional use with the safety biopsy instrument of FIGS. 2-5C;

FIG. 11 depicts another embodiment of a cutting depth setting device for optional use with the safety biopsy instrument of FIGS. 2-5C;

FIG. 12 depicts a cutting depth setting device for use with the safety biopsy instrument of FIGS. 6A-8;

FIG. 13A shows an additional embodiment of the disclosed safety biopsy instrument with a removable and disposable retractable blade cartridge;

FIG. 13B is a section view of the safety biopsy instrument of FIG. 13A with the blade retracted within the removable cartridge;

FIG. 13C is a section view of the safety biopsy instrument of FIGS. 13A-13B with the blade exposed;

FIG. 14 is a partial section view depicting removal of the blade cartridge;

FIG. 15A is an enlarged section view of the removable cartridge in the compressed state prior to disengagement with the handle portion;

FIG. 15B is an enlarged section view of the removable cartridge in the natural uncompressed state; and

FIG. 15C is a top view of the removable cartridge from above the locking projection.

### DETAILED DESCRIPTION OF THE PREFERRED EMBODIMENT

With reference to the drawing wherein like numerals represent like parts throughout the Figures, a safety biopsy instrument is disclosed.

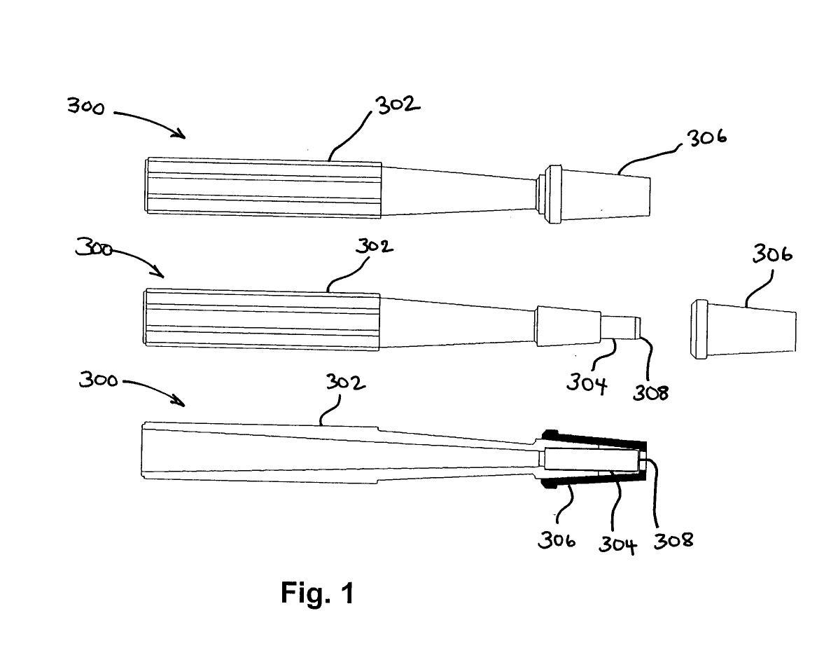

As a predicate to disclosure of the safety biopsy instrument, FIG. 1 depicts a disposable biopsy instrument 300 as known in the prior art. As can be seen, the instrument 300 comprises a tubular blade 302 attached to a handle or base 304, and a removable cover 306. The removable cover 306 initially conceals the sharp edge 308 of the blade 302. A user can manually remove the cover 306 to expose the edge 308 and use the instrument to make an incision. After use, the cover 306 can be placed back over the blade 304 to conceal the edge 308. Known biopsy instruments like that depicted in FIG. 1 present a potential safety hazard in the event that the cover 306 is lost or accidentally disengages with the base 304, thus exposing the sharp blade edge 308.

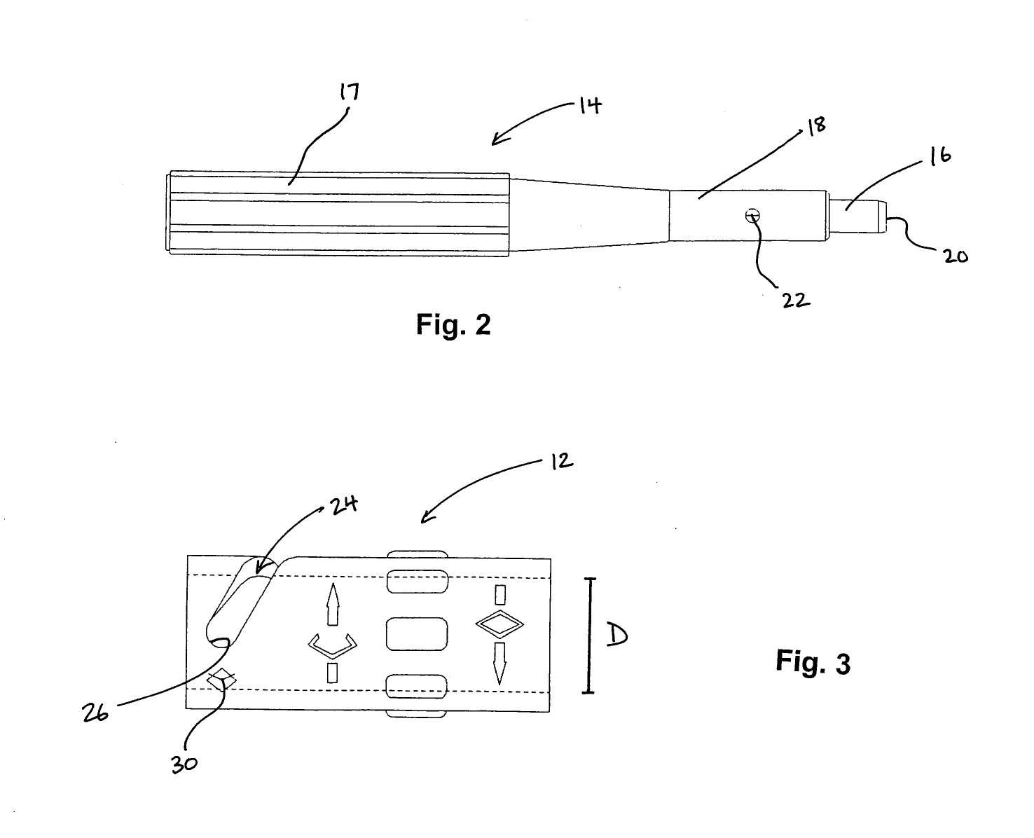

FIGS. 2-5C show a first embodiment of a safety biopsy instrument 10 with a permanently attached shield 12. FIG. 2 is an elevation view of the instrument's body 14 attached to a tubular blade 16 prior to engagement with the shield 12. As can be seen, the body 14 has an axial length, a proximal handle portion 17 and a generally cylindrical distal portion 18. The blade edge 20 extends forward of the distal end 18. The body distal end 18 has a radially projecting pin 22.

FIG. 3 is an elevation view of the shield 12 prior to engagement with the body 14. The shield is generally tubular in shape with an inner diameter D that corresponds generally to the outer diameter of the body distal portion 18. As can be seen, the shield 12 is fit with a helical aperture that defines a track 24. The helical aperture has closed proximal and distal ends, 26 and 28 respectively. The outer surface of the shield 12 can be fit with serrations or a similar fitting to improve friction for a user to grip.

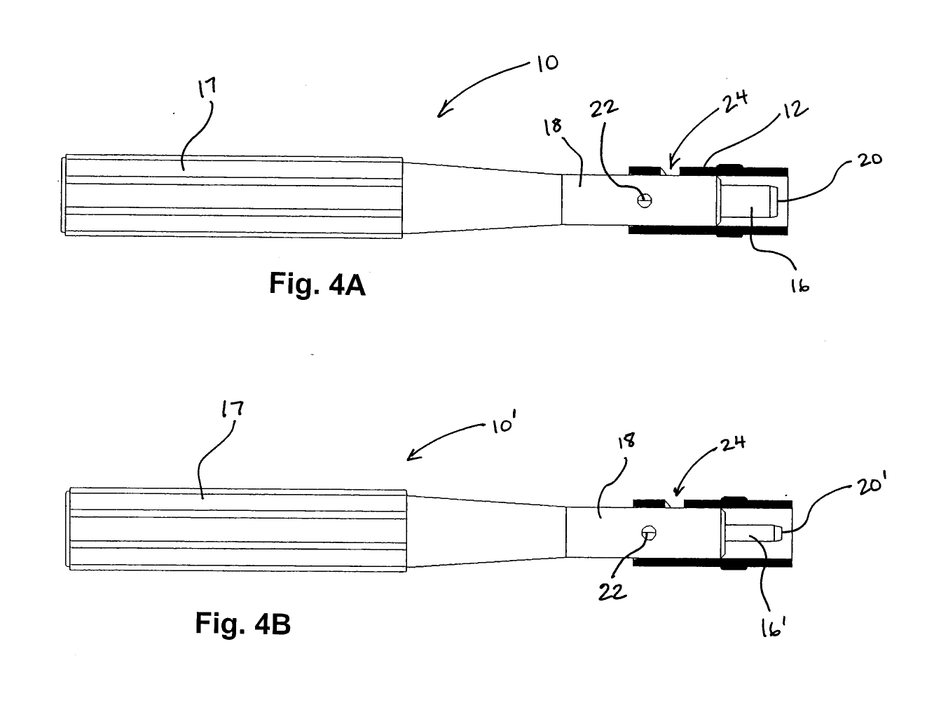

FIGS. 4A and 4B are partial section views of the instrument engaged with a shield 12 in the closed position. As shown, the shield 12 can be employed in the inventive instrument regardless of the diameter of the tubular blade.

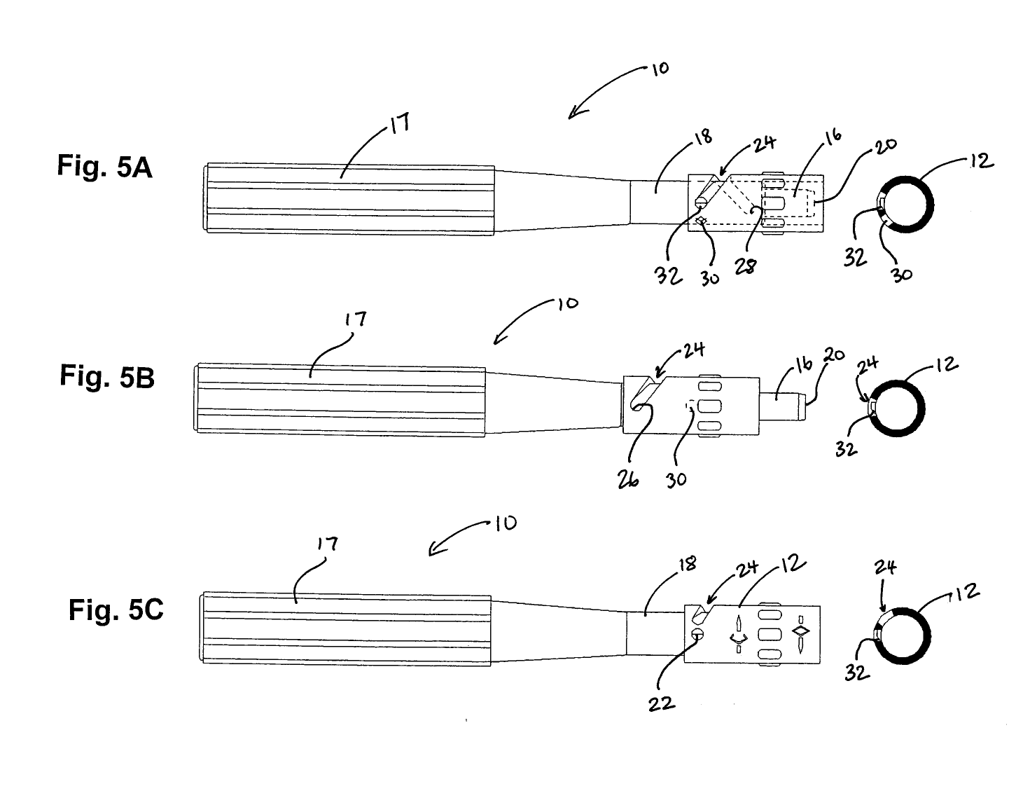

As can be seen in FIGS. 5A and 5B, the shield 12 and body 14 are engaged via receipt of the lateral pin 22 by the helical track 24. FIG. 5A shows the instrument 10 with the shield 12 engaged and in an initial closed position with the pin 22 positioned at the proximal end 26 of the track. In the initial closed position, the shield 12 extends forward of the blade edge 20, protecting a user against accidental injury from the sharp edge 20. Typically, the instrument 10 will be packaged in a sterile sealed pouch with the shield 12 in the initial closed position of FIG. 5A. As shown in FIGS. 5A and 5C, the shield 12 is also fit with a lateral bore 30 slightly rearward of the proximal end 26 of the helical track 24.

With reference to FIG. 5B, the blade edge 20 can be exposed by rotation of the shield 12 relative to the base 14 (counter-clockwise in the depicted embodiment). This rotation causes the shield 12 to travel rearwardly relative to the base 14 via the pin 22 positioned within the helical track 24. The closed distal end 28 of the track prevents additional rearward movement of the shield 12 once the pin 22 and distal end 28 meet (see FIG. 5B). Once the instrument 10 is in the exposed configuration of FIG. 5B, a user can make an incision with the blade edge 20 and extract a sample of tissue. Alternatively, a tissue sample can be taken by first pressing the distal end of the shield 12 against the tissue and then rotating the body 14, thereby extending the blade edge 20 past the shield 12 and simultaneously making an incision.

Once the instrument 10 has been used, reverse rotation (clockwise in the FIGS. 5A-5C embodiments) of the shield 12 relative to the body 14 causes the shield 12 to travel distally until the sharp blade edge 20 is concealed again. The shield 12 can be locked in the final closed position via engagement of the pin 22 within the lateral bore 30, thus preventing re-exposure of the sharp blade edge 20 via inadvertent rotation of the shield 12. Once the shield 12 is rotated into the locked position depicted in FIG. 5C, the instrument 10 can be disposed of safely. As can be seen, this embodiment features a pin 22 with a bevelled edge 32. The bevelled edge 32 facilitates continued rotation of the shield 12 past the position with the pin 22 at the closed proximal track end 26. Thus, the shield can easily be positioned in the locked position with the pin 22 within the bore 30.

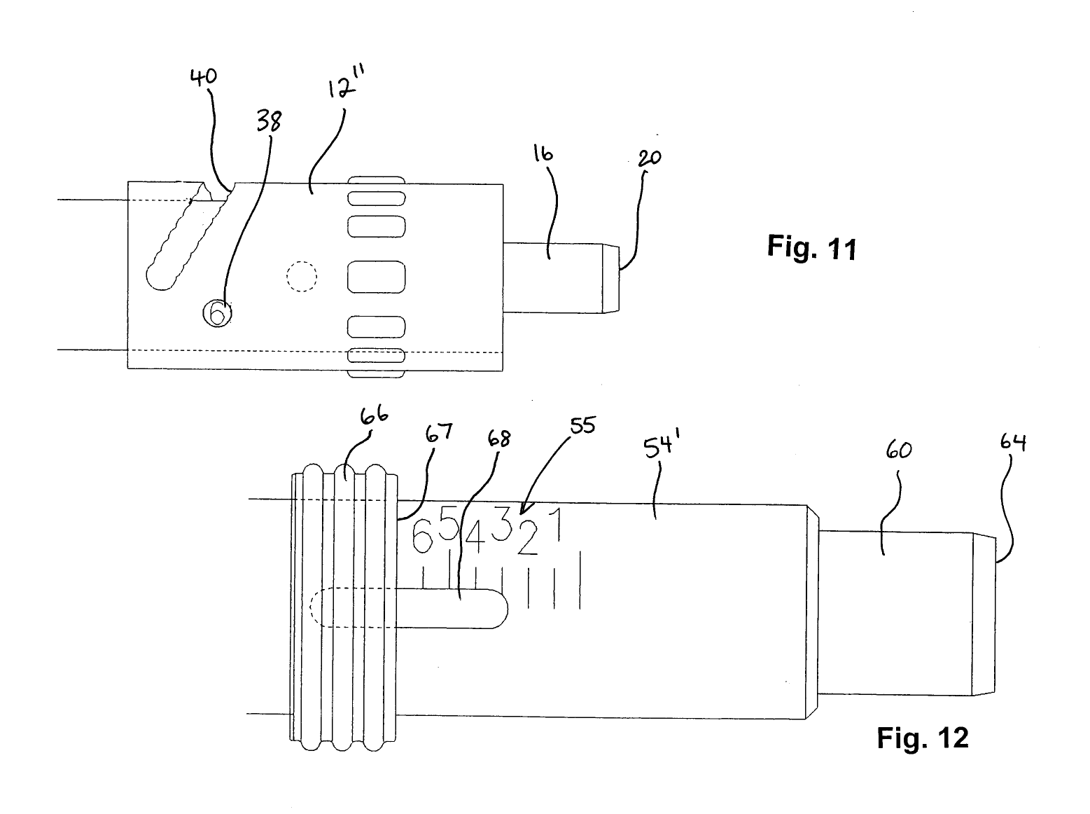

This embodiment of the instrument 10 can also be equipped with depth setting indicators such as those shown in FIGS. 10 and 11. FIG. 10 shows an embodiment of indicator wherein the body measurement markings 34 that correspond to particular depths and the shield 12′ has a reference line 36. A user can thus rotate the shield 13 until the reference line 36 reaches a desired depth measurement. The distal end of the shield will prevent the blade from cutting below the desired depth. FIG. 11 depicts an alternate embodiment of a depth-cutting indicator, wherein the shield 12″ has a small opening 38 that aligns with numbers fixed on the body corresponding to depths. Also shown in FIG. 11 is a helical track fit with grooved edges 40 to help prevent inadvertent rotation of the shield. Clearly, incorporation of the grooved edges is not limited to the particular embodiment of shield depicted in FIG. 11.

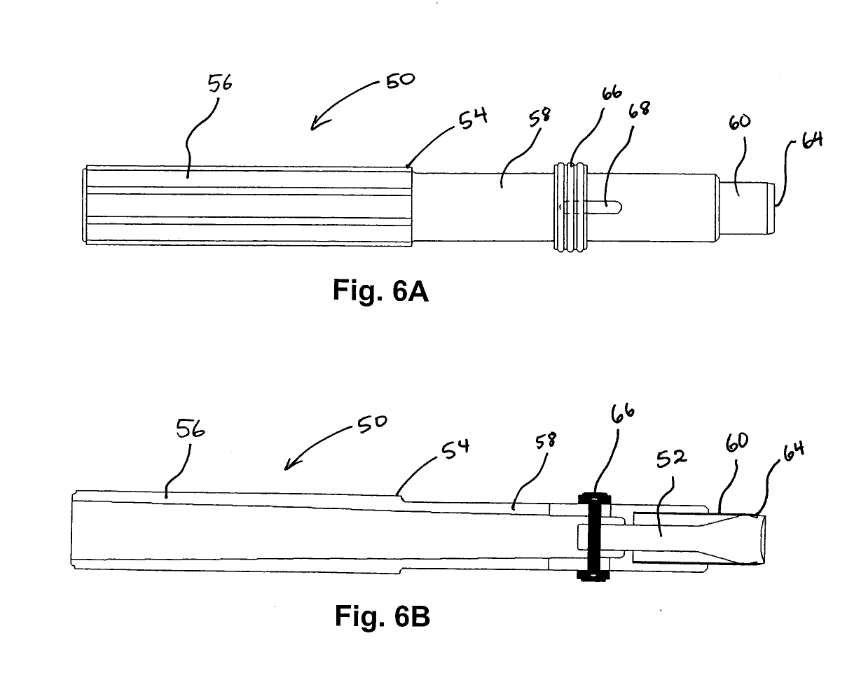

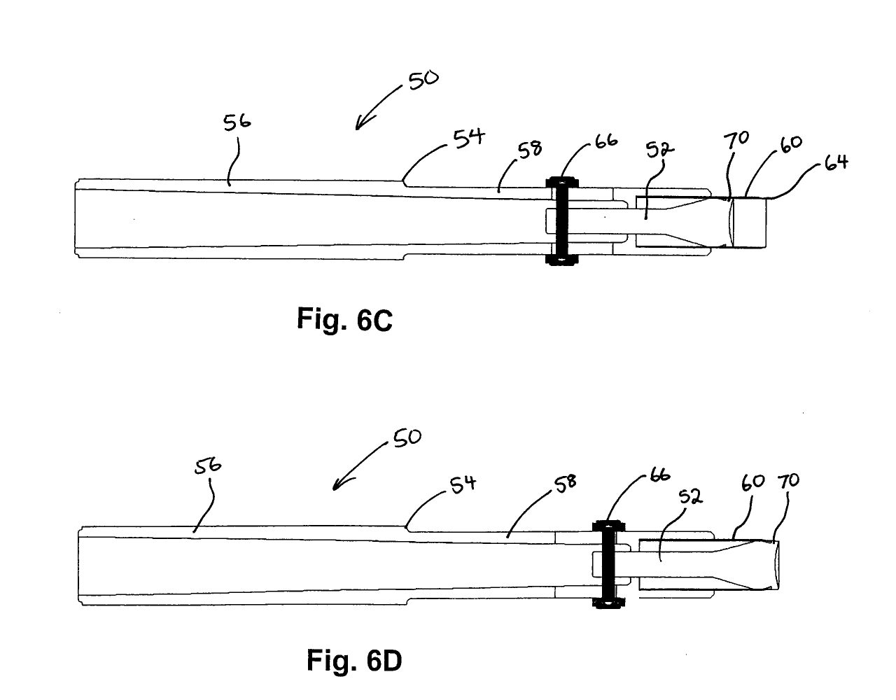

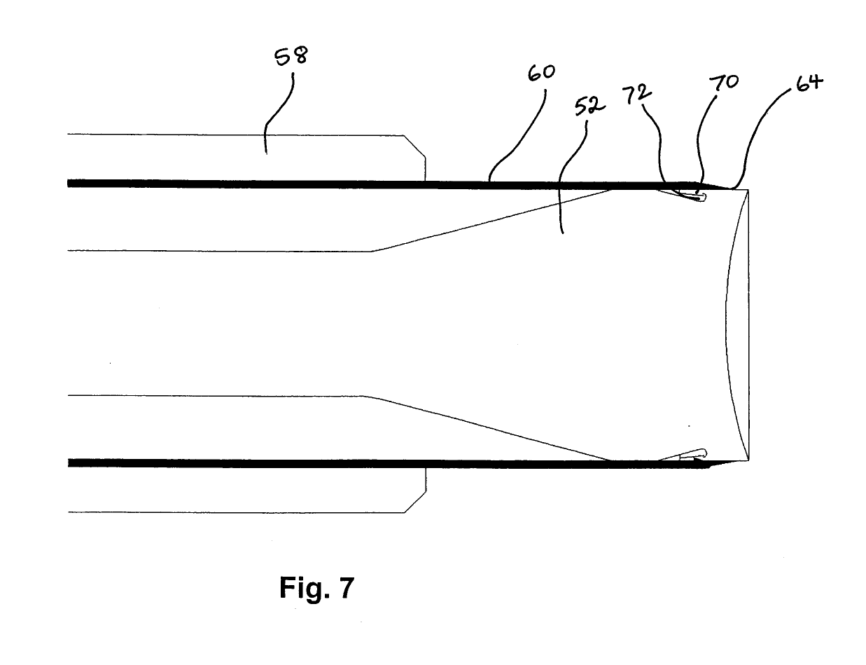

FIGS. 6A-6D depict another embodiment of the disclosed safety biopsy instrument 50 with a protective plug 52, rather than a rotatable shield. Like the previous embodiment, the instrument 50 has an axially extending body 54 with a proximal handle portion 56 and a distal portion 58. A tubular blade 60 defining an internal bore 62 is attached to the body 54 with the sharp blade edge 64 extending forward of the body's distal end. Rather than a shield that prevents blade exposure by surrounding the blade, this embodiment has a plug 52 positioned within the blade bore 62. FIG. 6B is a section view of the instrument 50 with the plug 52 extending slightly beyond the blade edge 64. FIG. 7 is an enlarged section view of the distal end of the device showing the sleeve 52 extending beyond the blade edge 64 in detail. Typically, the instrument 50 is packaged in a sterile pouch in this initial protected position depicted in FIGS. 6B and 7.

In this embodiment, the protective plug 52 is axially displaceable via a manually accessible actuator 66 that is attached to the plug 52. Here, at least the proximal end of plug 52 is positioned within the hollow body 54. The actuator 66 is attached near the plug's proximal end and extends out of an axial track 68 in the body 54. The blade edge 64 can be exposed by manually shifting the actuator 66 proximally, causing the plug 52 to retract within the blade bore 62. FIG. 6C shows the instrument 50 with the plug 52 in the retracted position and the blade edge 64 exposed for use.

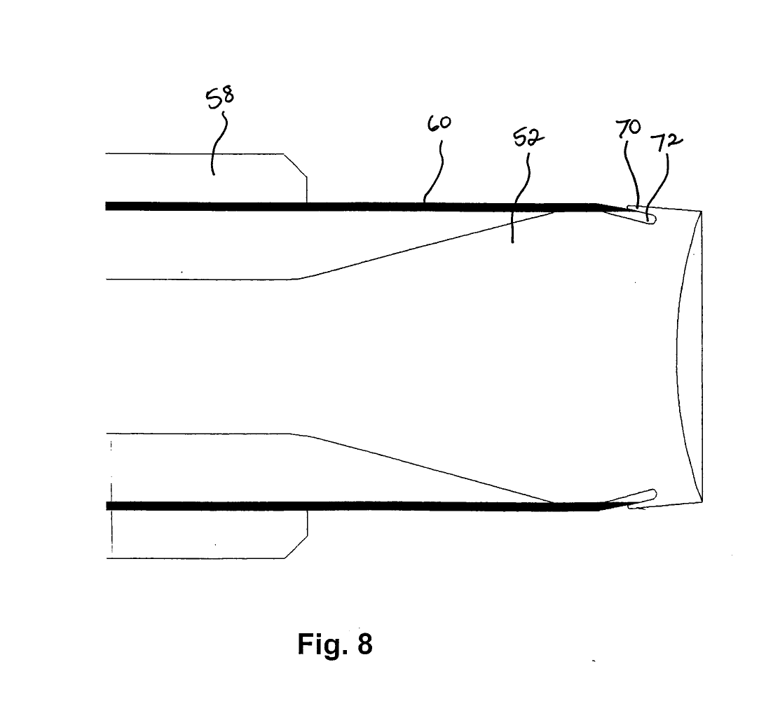

After the instrument 50 is used to make an incision, the plug 52 can be displaced forwardly via manual shifting of the actuator 66 distally. As seen most clearly in FIGS. 7 and 8, the distal end of the plug 52 is fit with an outward radial lip 70 that defines a radial notch 72. The plug 52 is initially positioned with the lip 70 tucked within the blade bore 62 to allow retraction of the plug 52 into the bore. After use of the instrument, the plug 52 can be extended into an axially locked position with the plug lip 70 forward of the blade edge 64 such that the blade edge engages with the radial notch 72. This engagement mechanically prevents re-exposure of the blade edge 64 because the plug 52 cannot travel proximally. The instrument can then be safely disposed of.

This embodiment can also be equipped with a cutting depth indicator, an example of which is shown in FIG. 12. Here, the body 54′ is fit with lines and measurements 55 corresponding to particular desired cutting depths and the front edge 67 of the actuator acts as a measurement marker. A user can shift the actuator 66 rearward until the front edge 67 reaches a desired depth measurement. The plug 52 will be positioned within the blade bore 62 at the appropriate axial location to prevent the blade edge 64 from passing the desired depth of tissue. This embodiment can also be fit with grooved edges in the axial body track 68 to improve depth accuracy and minimize risk of inadvertent movement of the actuator (grooved edges not depicted). Like with the previous embodiment, relative size and shape of the instrument components are not limited to those depicted in FIGS. 6A-8.

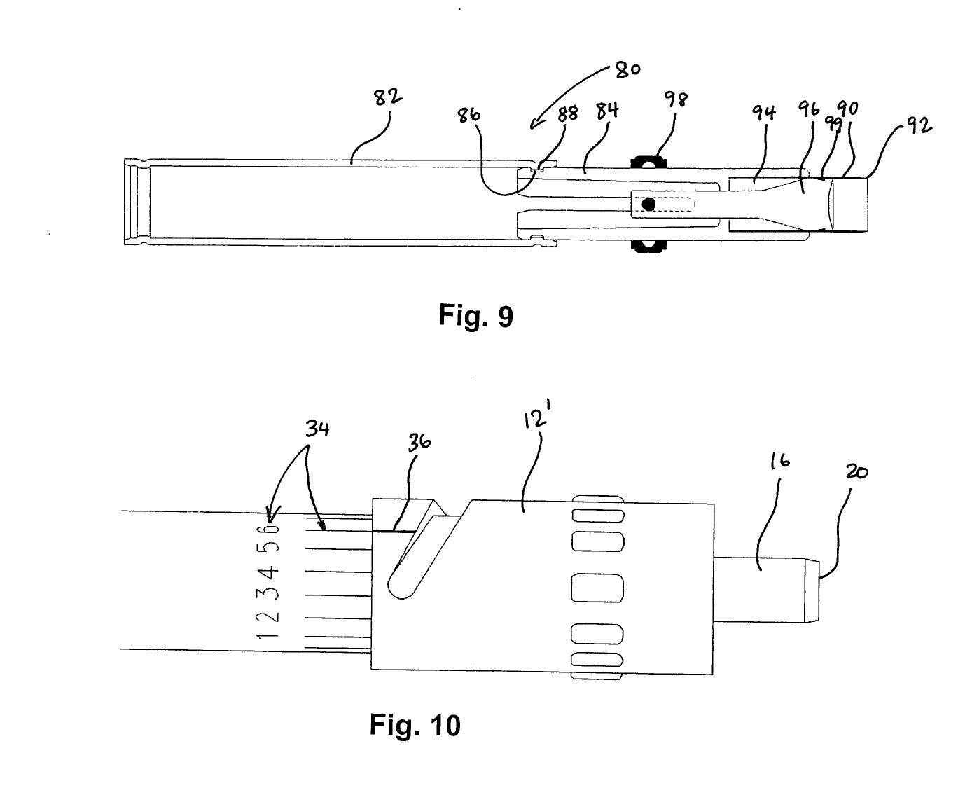

A variation of the blade plug embodiment of the disclosed instrument is shown in FIG. 9. The instrument 80 comprises a reusable handle 82 and a separate disposable blade cartridge 84. The handle 82 can be reused and blade cartridges 84 can be packaged individually in sterile pouches. The depicted blade cartridge 84 is configured for attachment to the handle 82 via a notch 86 that cooperates with a projection 88 in the handle. This notch/projection attachment configuration is not limiting. The blade cartridge 84 is configured like the previous embodiment of the instrument 50, having a tubular blade 90 with a sharp edge 92 extending forward of the cartridge distal end. The tubular blade 90 defines a bore 94 within which a protective plug 76 is positioned. Exposure of the blade edge 92 for use is accomplished by manual rearward actuation of the externally extending actuator 98. Like the previous embodiment, the blade edge 92 is concealed and locked for safe disposal by reciprocating the actuator forward and extending the radial lip 99 on the plug 96 past the blade edge 92. In this embodiment, the blade cartridge 84 is removed from the handle 82 and disposed of. The handle portion 82 can be kept and reused via attachment of a new suitable blade cartridge.

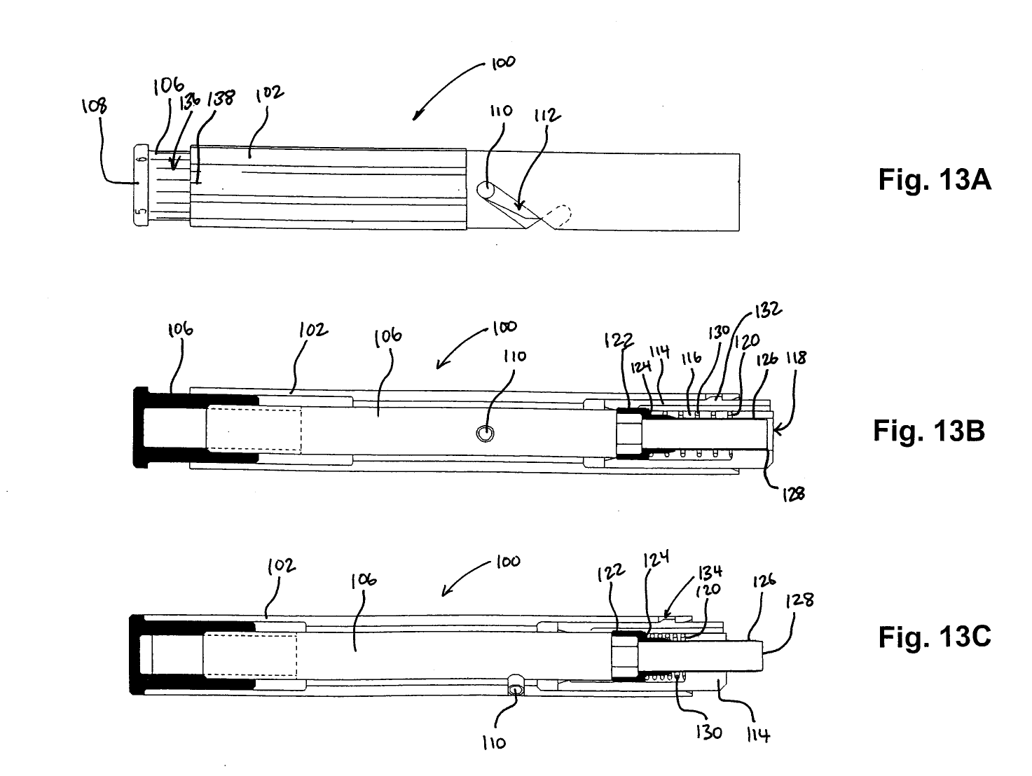

Yet another embodiment of the disclosed biopsy instrument is shown in FIGS. 13A-15C. This instrument 100 comprises a reusable handle 102 and disposable blade cartridge 104, both having axial lengths. A body 106 is disposed coaxially within the handle 102 with a proximal portion of the body 106 extending rearward of the handle proximal end 108. The body 106 is fit with a laterally extending pin 110 that is positioned between the closed proximal and distal ends of a helical track 112 in the handle 102.

The removable blade cartridge 104 has a generally cylindrical housing 114 that defines a cavity 116 with a distal opening 118. The housing has a front shoulder 120 that extends radially into the cavity 116. The housing base 122 defines a rear wall 124. A tubular blade 126 is coaxially attached to the base 122 with the sharp blade edge 128 extending forward of the wall 124. A return member 130 is positioned within the housing 114 axially between the front shoulder 120 and rear wall 124. The return member 130 is configured to bias the blade edge 128 away from the distal opening 118.

The housing base 122 is configured for engagement with the distal end of the body 106. The Figures depict a nonlimiting example of such engagement wherein the base 122 defines a hexagonal recess that cooperates with a hexagonal projection on distal end of body 106.

The blade cartridge 104 can engage with the body 106 via manual insertion of the cartridge 104 into the distal end of the hollow handle 102. In this embodiment, the cartridge housing 114 is fit with a laterally projecting pin 132 that engages with a recess 140 on the inner surface of the handle 102 to lock the cartridge in place prior to use. The blade cartridge 104 is configured so that the blade edge 128 is positioned rearward of the distal opening 118 when the cartridge is in the initial relaxed position depicted in FIG. 13B. Rotation of the handle 102 relative to the body 106 in one direction (clockwise in FIGS. 13A-13C) causes the body 106 to move forward relative to the handle 102 and compress the return member 130. Compression of the return member 130 causes the blade edge to travel forward relative to and eventually past the distal opening 118, exposing the edge 128 for use (FIG. 13C). In the Figures, the return member 130 is a helical spring. However, any known other component that can bias the blade edge from the distal opening and be compressed can be substituted, such as an elastic or rubber insert.

As seen in FIG. 13A, the instrument 100 can also employ a cutting depth indicating system. In this embodiment, the proximal end of the body 106 has measurement markings 136 that correspond to particular depths and the handle 102 has a reference line 138. A user can rotate the handle until the reference line 138 reaches the measurement of the desired cut depth and thus control the axial length of the blade 126 that is exposed. Alternatively, a user can make an incision by first pressing the distal end of the instrument 100 against a patient's tissue with the instrument in the initial relaxed position, and then rotating the body 104, thus extending the blade edge 128 directly into the tissue.

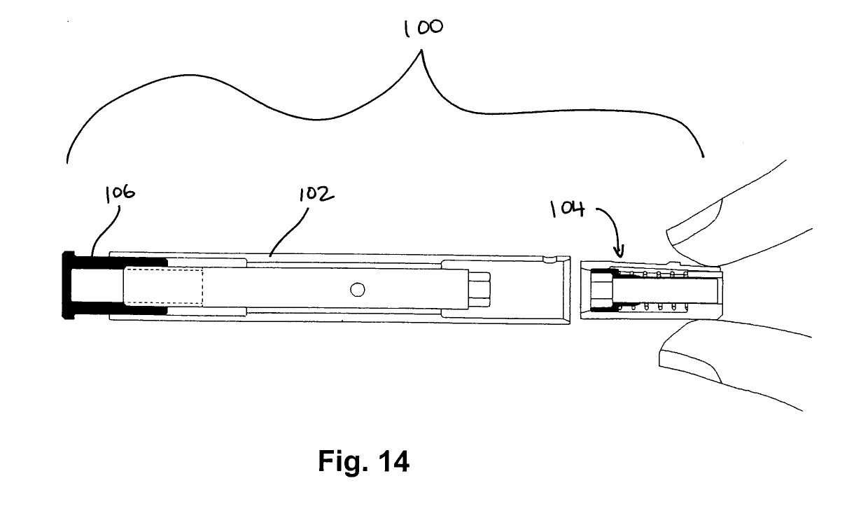

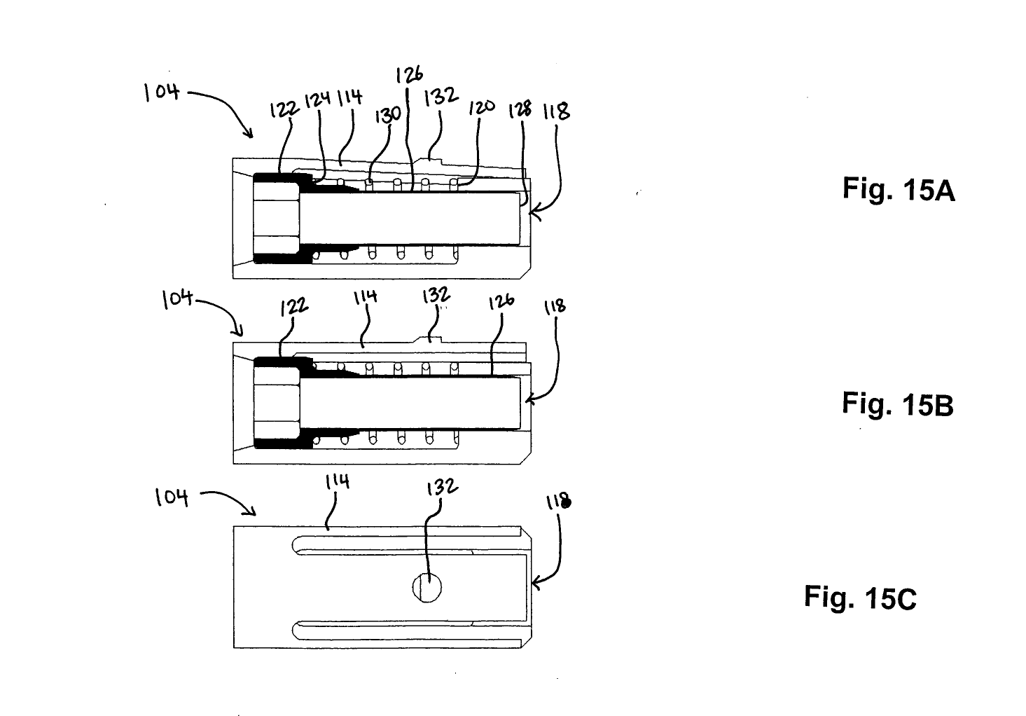

After an incision is made with the instrument 100, the blade edge 128 can be returned within the distal opening 118 by reverse rotation of the handle 102 relative to the body 106. Once the instrument 100 has been returned to the relaxed closed position with the blade edge 128 concealed by the cartridge housing 114, the cartridge 104 can be safely removed from the handle 102 and disposed of (FIG. 14). With reference to FIG. 15A, this embodiment of the disposable cartridge 104 has a housing 114 that is at least partially compressible. The compressibility facilitates removal of the cartridge 104 from the handle 102 by allowing disengagement of the locking pin 132 from the handle recess 134. FIG. 15A shows the housing 104 in the compressed state ready for removal from the handle 102. FIG. 15B shows the housing 104 in the natural uncompressed state. FIG. 15C shows a view of the compressible housing 104 directly above the locking pin 132.

While a preferred embodiment has been set forth for purposes of illustration, the foregoing description should not be deemed a limitation of the invention herein. Accordingly, various modifications, adaptations and alternatives may occur to one skilled in the art without departing from the spirit of the invention and scope of the claimed coverage.

1. A medical instrument for use in incising and extracting tissue, comprising:
an axially extending body with distal and proximal ends;
a tubular blade coaxially engaged with said body with a sharp edge that extends forward of said body distal end;
a protection member engaged with the body such that at least one of the protection member and blade edge are axially displaceable relative to each other from a position with the blade edge concealed by the protection member to a position with the blade edge exposed, and then to a locked concealed position wherein the protection member and blade edge are axially locked relative to each other.

2. A medical instrument for incising and extracting tissue, comprising:
an axially extending body with distal and proximal ends;
a tubular blade defining a bore coaxially engaged with said body with a sharp circular edge that extends forward of said body distal end;
a protective plug disposed within the bore configured for axial displacement relative to the blade edge, the protective plug having proximal and distal ends; wherein
the protective plug is manually axially displaceable from an initial position with the plug distal end extending forward of the blade edge to a retracted position with the plug distal end positioned within the bore and the blade edge being exposed, to an axially locked position with the plug distal end extending forward of the blade edge.

3. The medical instrument of claim 2, wherein the protective plug is lockable relative to blade edge in the locked position by an outwardly projecting lip on the plug distal end that extends radially beyond the diameter of the circular edge of the blade.

4. The medical instrument of claim 3, wherein in the locked position the lip engages with the blade edge to mechanically prevent distal movement of the plug.

5. The medical instrument of claim 2, wherein said base is hollow and defines a cavity and at least the proximal end of the plug is positioned within the base cavity, comprising an actuator attached to the protective plug and extending outside of said base cavity, the actuator being manually axially displaceable to effect axial reciprocation of the plug from the initial position to the retracted position to the axially locked position covering the blade edge.

6. The medical instrument of claim 2, wherein the position of the distal end of the plug in the initial position is distal to the position of the distal end of the plug in the retracted position and proximal to the position of the distal end of the plug in the axially locked position.

7. A medical instrument for incising and extracting tissue, comprising:
an axially extending body with distal and proximal ends disposed coaxially within a tubular handle, the handle having a helical track with closed proximal and distal ends between which a pin that extends radially from the body is positioned;
a removable blade cartridge configured for engagement the body within the tubular handle comprising
a generally cylindrical hollow housing defining a cavity, the housing having a distal opening;
a front shoulder extending radially into said cavity;
a tubular blade attached to a base that defines a rear wall, the blade having a sharp edge that extends forward of said rear wall;
a return member disposed within the housing engaged between the rear wall and front shoulder to bias the blade edge from the distal opening;
wherein
the distal end of the body is configured to engage the cartridge base so that the blade edge is positioned rearward of the distal opening when the pin is positioned at the proximal end of the helical track and rotation of the handle in a first direction relative to the base causes the base to move forward relative to the handle and cartridge housing, compressing the return member between the rear wall and front shoulder and causing the blade edge to travel distally to an exposed position forward of the distal opening until the pin meets the distal track end, then reverse rotation of the handle relative to the base causes the blade edge to travel proximally to a retracted position rearward of the proximal opening until the pin meets the proximal track end.

8. The medical instrument of claim 7 wherein the cartridge housing comprises a radially extending tooth configured for engagement with a recess in the handle when the cartridge is engaged with the distal end of the body.

9. The medical instrument of claim 8 wherein at least a portion of said cartridge housing is compressible.

10. A medical instrument for use in incising and extracting tissue, comprising:
an axial body with distal and proximal ends and an outer surface, at least a distal portion of said outer surface being generally cylindrical and having a laterally extending pin positioned distally of said proximal end;
a tubular blade engaged with said body with a sharp edge that extends forward of said body distal end;
a tubular shield with an outer surface and an inner diameter that corresponds generally to the diameter of said cylindrical distal portion of the body outer surface, the shield being engaged with the body coaxially via a helical aperture in the shield that defines a track that receives said laterally extending pin, the track having closed distal and proximal ends, and a bore extending laterally through the shield proximate said track distal end; wherein
the shield extends beyond said sharp edge when the pin is at the distal end of the track and rotation of the shield in a first direction relative to the body causes the shield to travel distally relative to the body, eventually exposing the sharp edge, until the proximal track end meets the pin, and reverse rotation of the shield relative to the body causes the shield to travel proximally until the pin engages with the lateral bore to lock the shield in a position concealing the sharp edge.

11. The medical instrument of claim 2, comprising a reusable handle that is attachable and detachable to the body.

12. The medical instrument of claim 11, wherein the handle extends from a proximal end to a distal end that is configured to engage with the body proximal end, the handle and body being generally coaxial when engaged.

13. The medical instrument of claim 5, wherein the body comprises an axial opening with closed proximal and distal ends, the axial opening defining a track through which the actuator extends, the actuator being axially displaceable between the proximal and distal end of the track.

14. The medical instrument of claim 13, wherein the track has two opposite axially extending lateral edges with grooves that engage the actuator.

15. The medical instrument of claim 2, comprising markings on the outer surface of the body that correspond to tissue incision depths so that when the actuator is axially displaced to a particular marking, the plug distal end is axially positioned within the bore to contact the tissue at a position that prevents the blade edge from incising the tissue deeper than a particular depth.

16. The medical instrument of claim 7, wherein the return member is a helical spring positioned within the housing substantially coaxially to the tubular blade.

17. The medical instrument of claim 7, wherein the helical track in the handle has opposite edges extending between the track proximal and distal ends, the opposite edges being fit with grooves that engage the pin.

18. The medical instrument of claim 7, wherein the proximal end of the body extends proximally from the proximal end of the handle.

19. The medical instrument of claim 7, comprising a mechanism for measuring the approximate axial distance between the blade edge and the housing distal opening when the instrument is in the exposed position thereby preventing an incision of tissue that is deeper than said axial distance.

20. The device of claim 7, wherein the base of the blade cartridge and the blade are configured so that rotation relative to the cartridge housing is allowed, the cartridge housing is rotationally fixed within the handle when engaged therein, and the base of the blade cartridge defines a non-cylindrical recess configured to receive a distal portion of the body so that rotation of the body relative to the handle causes the base and blade to rotate relative to the handle.
